(19) United States
(12) Patent Application Publication
(10) Pub. No.: US 2006/0014278 A1
(43) Pub. Date: Jan. 19, 2006

(54) Method for programmed differentiation of stem cells

(76) Inventor: Jaspal S. Khillan, Cherry Hill, NJ (US)

Correspondence Address: LICATLA & TYRRELL P.C., 66 E. MAIN STREET, MARLTON, NJ, 08053, US

(21) Appl. No.: 10/534,562

(22) PCT Filed: Dec. 10, 2003
(86) PCT No.: PCT/US03/39346

§ 371 Date: May 9, 2005

Related U.S. Application Data
(60) Provisional application No. 60/432,388, filed on Dec. 10, 2002.

(51) Int. Cl. C12N 5/08; C12N 15/02

(52) U.S. Cl. 435/366; 435/450

(57) ABSTRACT

Provided is a method for programming the differentiation of stem cells and for the identification of the signals responsible for cell lineage establishment of differentiated cells.

### BACKGROUND OF THE INVENTION

Stem cells have two important characteristics that distinguish them from other types of cells. First, they are unspecialized cells that renew themselves for long periods of time through cell division. Second, under certain physiologic or experimental conditions, they can be induced to become cells with special functions such as the beating cells of the heart muscle or the insulin-producing cells of the pancreas. Scientific experiments have been conducted primarily on two kinds of stem cells from animals and humans, namely, embryonic stem cells and adult stem cells, also known as somatic stem cells. Stem cells are useful to study gene functions and regulation, human diseases, and targeted cell differentiation. When unspecialized stem cells give rise to specialized cells, the process is called differentiation. Differentiation of stem cells has major implications in clinical applications for curing degenerative diseases in humans. The differentiation of embryonic stem cells into embryoid bodies has been used to study the differentiation of the embryonic stem cells into different types of cells (W. Mueller-Klieser, Am. J. Physiol. 273, C1109 (1997)). Trans-differentiation of somatic stem cells into cells different from the parental lineage has also been reported (D. L. Clarke et al., 2000 Science 288:1660; R. Galli et al., 2000 Nat. Neurosci. 10:986; R. L. Rietze et al., 2001 Nature 412:736; G. Condorelli et al., 2001 Proc. Natl. Acad. Sci. U.S.A. 98:10733). However, more recent reports contradict the ability of somatic stem cells to differentiate into cells different from the parental lineage, as it appears that fusion of the somatic stem cells with embryonic stem cells may have resulted in trans-differentiation properties (Ying et al. 2002 Nature 416:545; Terada et al., 2002 Nature 416:542.

Pluripotent embryonic stem cells are the most versatile cells with wide potential to produce all types of cells. However, so far it has not been possible to control their differentiation. Programmed differentiation of pluripotent embryonic stem cells into specific lineages is the most limiting step in exploiting their potential for clinical applications for degenerative diseases such as Alzheimer's disease, Parkinson's disease, osteogenesis imperfecta, osteoarthritis, diabetes, or heart disease, as well as for tissue engineering and repair. Since pluripotent embryonic stem cells have infinite capacity for self-replication, they are potentially an unlimited source of cells for therapies in humans. WO 200210347 describes mapping a pathway of a population of embryonic cells wherein the embryonic cells are exposed to an exogenous factor, and the gene expression products characteristic of a particular cell type are measured. The exogenous factor is a growth factor, an interleukin, a nerve growth factor or a retinoic acid. The differentiated cells are neuronal cell types which have neuronal processes. Kramer et al., 2000 Mechanisms of Development 92:193-205, disclose that differentiation of mouse embryonic stem cells into chondrocytes can be modulated by members of the transforming growth factor beta family (TGF-beta(1), BMP-2 and BMP-4).

The present invention provides a method for programming the differentiation of stem cells using precursor cells from embryos.

### SUMMARY OF THE INVENTION

The present invention provides a method of programming the differentiation of stem cells comprising contacting stem cells with precursor cells to form a mixture whereby the stem cells become primed and then allowing the primed stem cells to differentiate.

The present invention further provides a method to identify signals responsible for cell lineage establishment comprising defining the prerequisite conditions to the differentiation of stem cells into their preferred lineage, and determining which prerequisite conditions are signals responsible for cell lineage establishment.

### DETAILED DESCRIPTION

Stems cells are cells with the ability to divide for indefinite periods in culture and to give rise to specialized cells. Stem cells differ from other kinds of cells in the body. All stem cells, regardless of their source, have three general properties: they are capable of dividing and renewing themselves for long periods; they are unspecialized; and they can give rise to specialized cell types. A somatic stem cell is an undifferentiated cell found among differentiated cells in a tissue or organ. A somatic stem cell can renew itself, and can differentiate to yield the parental cell types of the tissue or organ. The primary roles of adult stem cells in a living organism are to maintain and repair the tissue in which they are found.

The present invention provides a method for programming the differentiation of stem cells comprising contacting the stem cells with precursor cells to form a mixture whereby the stem cells become primed. The stem cells may be either somatic or embryonic. In a preferred embodiment, the stem cell is an embryonic stem cell. A selection compound may be added to the mixture, but is not required. The selection compound may be a marker, or an appropriate drug used to identify the primed cells, or those stem cells which have made sufficient contact with the precursor cells and are capable of differentiation. After selection, the primed cells may be either allowed to differentiate in vitro with cell specific growth factors, such as angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor-alpha, cytokine-induced neutrophil chemotactic factor 1, cytokine-induced neutrophil, chemotactic factor 2-alpha, cytokine-induced neutrophil chemotactic factor 2-beta, beta-endothelial cell growth factor, endothelia 1, epidermal growth factor, epithelial-derived neutrophil attractant, fibroblast growth factor 4, fibroblast growth factor 5, fibroblast growth factor 6 fibroblast growth factor 7, fibroblast growth factor 8, fibroblast growth factor b, fibroblast growth factor c, fibroblast growth factor 9, fibroblast growth factor 10, fibroblast growth factor acidic, fibroblast growth factor basic, glial cell line-derived neutrophil factor receptor-alpha-1, glial cell line-derived neutrophil factor receptor-alpha-2, growth related protein, growth related protein-alpha, growth related protein-beta, growth related protein-gamma, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor-alpha, nerve growth factor, nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor-alpha, platelet derived growth factor receptor-beta, pre-B cell growth stimulating factor, stem cell factor, stem cell factor receptor, transforming growth factor-alpha, transforming growth factor-beta, transforming growth factor-beta-1, transforming growth factor-beta-1-2, transforming growth factor-beta-2, transforming growth factor-beta-3, transforming growth factor-beta-5, latent transforming growth factor-beta-1, transforming growth factor-beta-binding protein I, transforming growth factor-beta-binding protein II, transforming growth factor-beta-binding protein III, tumor necrosis factor receptor type I, tumor necrosis factor receptor type II, urokinase-type plasminogen activator receptor, vascular endothelial growth factor, and chimeric proteins and biologically or immunologically active fragments thereof; or may be injected directly into an animal system for in vivo integration.

A “precursor cell” is a cell which will program or prime a stem cell to differentiate into a specific cell lineage. In a preferred embodiment, the precursor cell is a limb bud cell (LBC) which will program or prime a stem cell to differentiate into a chondrocyte, other examples of suitable precursor cells include but are not limited to hematopoetic precursors, osteogenic precursors and islet precursors. Precursor cells are likely to die off naturally as stem cells differentiate and expand. Thus, the precursor cells are unlikely to be present in advanced stages of differentiation. The differentiated cell is derived from primed cells which may differentiate in vivo or in vitro.

Adult stem cells are generally limited to differentiating into different cell types of their tissue of origin. However, if the starting stem cells are derived from the inner cell mass of the embryo, they can generate many cell types of the body derived from all three embryonic cell types: endoderm, mesoderm and ectoderm. Stem cells with this property are said to be pluripotent. Embryonic stem cells are one kind of pluripotent stem cell. Thus, pluripotent embryonic stem cells can be differentiated into many specific cell types. For example, using precursor cells from the limb bud of a developing embryo, differentiation of embryonic stem cells into chondrocytes, or cartilage producing cells was obtained. Embryonic stem cells which differentiate into chondrocytes express genes specific for cartilage such as collagen type II and proteoglycans. Since the embryo is a potential source of all types of precursor cells, it is possible to differentiate embryonic stem cells into other lineages by providing the appropriate precursor cells or signals to embryonic stem cells. Somatic stem cells also have major advantages, for example, using somatic stem cells allows a patient's own cells to be expanded in culture and then reintroduced into the patient. The use of the patient's own adult stem cells would allow the patient to be treated without rejection of the cells by the immune system. This represents a significant advantage as immune rejection is a difficult problem that can only be circumvented with immunosuppressive drugs.

Pluripotent R1 embryonic stem cells, which contain neomycin phosphoribosyl transferase (neo) gene and enhanced green fluorescence (EGFP) gene, were cultured as micromass cultures with 10 to 50 percent limb buds cells (LBC) from E10.5-11.5 embryos. The initial concentration of about 25 percent LBC was sufficient to achieve programmed differentiation of a high percentage of embryonic stem cells. Since cell-to-cell interaction plays a critical role in in vivo differentiation, this concentration may be optimized to provide maximum cell-to-cell contact. A direct contact between the cells appeared to be critical since the cultures in which stem cells were surrounded by the islets of LBC failed to exhibit the same differentiation characteristics. Irradiation of LBC prior to culture also did not cause differentiation suggesting that fully active cells are required for induction of differentiation. Since the doubling time of embryonic stem cells is about 8 to 10 hours, the percentage of LBC was expected to decrease significantly with time. For example, fluorescent activated cell sorter (FACS) analysis of cultures with 30 percent LBC revealed, that the percentage of these cells was reduced to about 12 percent after 24 hours.

The embryonic stem/LBC cultures exhibited morphology of uniform flat layers of cells within 48 hours as compared to similar cultures of pure embryonic stem cells that formed typical embryonic stem colonies. At days 6 to 8, the islands of condensed cells were seen dispersed throughout the culture, and formed prominent nodules by days 12 to 15. Each nodule contained between 100 and 200 cells, which is consistent with the typical features observed in micromass cultures of pure mesenchymal cells from limb buds. At certain areas, the nodules were surrounded by morphologically indistinct cells that failed to differentiate.

Normally, mesenchymal cells form condensations of cells after 3 days of culture. These condensations develop into nodules within 5 to 6 days and differentiate into chondrocytes within 10 to 12 days. The condensation of cells in embryonic stem/LBC cultures, however, were observed after about 6 to 7 days suggesting about a 3 to 4 day lag period for programming of the cells. It is believed that all of the nodules formed simultaneously suggesting programmed differentiation rather than a spontaneous differentiation of embryoid bodies. The control cultures of pure embryonic stem cells did not exhibit any of the above characteristics. To exclude the possibility that nodule formation occurred due to the aggregation of LBC in the mixed cultures, the same proportion of LBC was cultured with mitotically inactive irradiated primary fibroblasts. No chondrocyte condensations were observed after 14 days of culture which supports further the embryonic stem origin of the nodules in embryonic stem/LBC cultures.

Mesenchymal cells which are terminally differentiated into chondrocytes express genes such as collagen type II and sulphated proteoglycans. The chondrocyte nodules stain positive with alcian blue dye due to the presence of sulphated proteoglycans in the extracellular matrix. As expected, the nodules formed in 10 to 12 day, pure mesenchymal cultures stained positive with alcian blue dye. The nodules in 15 day embryonic stem/LBC cultures also showed strong positive staining with alcian blue dye. Similar to normal mesenchymal cultures, the staining was specific to the nodules only, indicating their differentiated characteristics. The control embryonic stem cultures failed to show any staining with the alcian blue dye. Alcian blue-stained nodules were also collected and analyzed by PCR. Amplification of a neomycin specific 495 bp band confirmed the embryonic stem origin of these cells. The cell cultures from four independent experiments showed that almost 80 to 90 percent of the cells differentiated into nodules and surrounding unicellular perichondrium-like cells. The high percentage of differentiated cells, brevity of the period for differentiation and failure of LBC to form nodules in control cultures, indicate that cell fusion is not the cause of observed differentiation.

Collagen type II, the major protein in the extracellular matrix of cartilage, is expressed in two forms due to alternate splicing of exon 2 of the mRNA. The pre-chondrogenic mesenchymal cells exclusively express the transcript with exon 2, whereas mature chondrocytes contain a transcript in which exon 2 is spliced out. To investigate the expression of the collagen type II gene, the embryonic stem cultures with LBC from wild type embryos were treated with 50 μg/ml G418 drug (Gibco, BRL) at day 4 to eliminate the LBC. At day 7, the cells were analyzed by reverse transcriptase PCR (RT PCR) using primers that amplify fragments specific for both spliced (285 bp) and unspliced (489 bp) transcripts. The cells from embryonic stem/LBC culture showed amplification of only a 489 bp fragment specific to the transcript with exon 2. Pre-chondrocytic mesenchymal cells at day 1 also showed the amplification of the same fragment which demonstrates the pre-chondrocyte nature of the cells. The absence of 285 bp fragment specific to the spliced transcript demonstrated the complete elimination of LBC by G418. On the other hand, day 7 parallel cultures of pure LBC amplified both of the fragments specific to spliced and unspliced transcripts, 285 bp and 489 bp respectively, indicating the differentiation of cells into chondrocytes. No amplification of the collagen type II gene specific fragments was observed in control embryonic stem cultures.

The chondroprogenitor cells express high levels of a high mobility group (HMG) transcription factor, sox 9, which parallels the expression of the collagen type II gene. RT PCR analysis revealed that day 7 embryonic stem cultures expressed high levels of sox 9 mRNA similar to that observed in day 1 and day 7 normal limb bud cultures, confirming the chondrogenic nature of the cells. No expression of sox 9 was observed in control embryonic stem cultures. All samples contained equal amounts of RNA as observed for HPRT MRNA used as a control. These data along with alcian blue dye staining, demonstrate the programming of embryonic stem cells into chondrogenic lineage.

To test the in vivo potential of primed cells, neo and EGFP positive embryonic stem cells derived from an FVB/N strain of mice were cultured with LBC. After four days of exposure, approximately 1×10⁶ cells were injected into the peritoneal cavity of 3 week old FVB/N mice. The animals were sacrificed after about 12 weeks to analyze the tissues for integration of cells by PCR of the fifteen different tissues analyzed, a neo specific gene amplification was observed only in the xiphoid cartilage. Amplification of the neo specific 495 bp fragment was observed in two different areas of the tissue demonstrating the integration of cells. No amplification of the fragment was observed in other cartilage tissues such as knee joints, nasal cartilage and the backbone. The absence of amplification of the neo specific fragment in these tissues may be due either to low abundance of fully programmed cells or dilution of the DNA by surrounding tissues or non-penetration of cells into these organs for which a direct injection of the cells may be carried out. Further, the exclusion of cells from other tissues indicates their predetermined nature into chondrogenic lineage. The finding that a brief exposure of stem cells to precursor cells is sufficient to determine their fate in vivo, has significant implications for clinical applications.

No additional growth factors were added to the programmed ES/LBC cell cultures suggesting that micromass cultures are sufficient to induce differentiation of the stem cells. To test the applicability of the present invention for other cell types, embryonic stem cells were cultured with precursor cells from other tissues of the embryo which resulted in cells with morphological characteristics of neuronal cells and cardiomyocytes. These studies demonstrate the potential application of this system to many cell types and to human embryonic stem cells as well, either from the existing lines or produced by therapeutic cloning as suggested by the integration of isologous embryonic stem cell line in FVB/N mice. Programmed differentiation of stem cells from mature tissues, however, by the same approach can be highly useful to avoid the controversies associated with human embryonic stem cells.

In addition, the present invention also provides a method for identifying signals responsible for cell lineage establishment, patterning and morphogenesis. The signals may be physical signals such as cell-to-cell contact, electrical signals between cells, or chemical agents. The signals can be identified as those conditions which are prerequisite to the differentiation of stem cells into their preferred lineages. The signals responsible for cell lineage establishment can be identified, by defining the prerequisite conditions to the differentiation of stem cells into their preferred lineage, and then determining which prerequisite conditions are signals responsible for cell lineage establishment. Once the signals responsible for cell lineage establishment are identified, the cell lineage can be controlled or modulated by the signals. The mouse embryo may have limitations to separate specific precursors, because development in this species occurs very rapidly due to short gestation period, i.e. about 19 to 21 days. Embryos from species with extended gestation periods such as rabbit or pig may be used. Pig embryos may be more acceptable for human studies since this species is being pursued extensively for xeno-transplantation.

The present invention provides a method by which differentiated human stem cells can be used for testing new therapeutic agents or drugs. In a preferred embodiment, the invention provides a method of identifying effective therapeutic agents or drugs by screening a test agent on a first differentiated human stem cell (test cell) and comparing the results obtained from a control agent applied to a second differentiated human stem cell (control cell) from the same cell line. The method provides contacting a test cell with a test agent and comparing the results obtained in the test cell with a control cell contacted with a standard. As one of skill in the art may appreciate, a standard may comprise an agent which illustrates a positive outcome (i.e., positive standard) or a negative outcome (i.e., negative standard) to which an unknown test agent may be compared and indexed. For example, new medications could be tested for safety on differentiated cells generated from human pluripotent cell lines. The present invention provides wide availability of differentiated somatic or pluripotent stem cells, and allows testing of potential therapeutic agents in a wide range of cell types. Additionally, by allowing scientists to precisely control the differentiation of stem cells into the specific cell types on which therapeutic agents will be tested, potential therapeutic agents can be screened effectively. Further, the cell line establishment and test conditions would be identical for the comparison of different drugs.

Perhaps the most important potential application of human stem cells is the generation of cells and tissues that could be used for cell-based therapies. Today, donated organs and tissues are often used to replace ailing or destroyed tissue, but the need for transplantable tissues and organs far outweighs the available supply Stem cells, directed to differentiate into specific cell types, offer the possibility of a renewable source of replacement cells and tissues to treat diseases including Parkinson's and Alzheimer's diseases, spinal cord injury, stroke, burns, heart disease, diabetes, osteoarthritis, and rheumatoid arthritis. The present invention is further illustrated by the following, non-limiting examples.

### EXAMPLE 1

### Preparation of Embryonic Stem Cells and Limb Bud Cells from Embryo

Embryonic stem cells were cultured over irradiated primary fibroblasts following standard procedures known in the art. All other cultures were carried out in DMEM medium with 10 percent fetal bovine serum. A single cell suspension was prepared by trypsinization for 5 minutes with trypsin-EDTA followed by pipetting several times. Mouse embryos were isolated from 11.0-11.5 day pregnant FVB/N females with day of mating as day 0.5. Pooled limb buds from several embryos were trypsinized in a 1:1 mix of PBS and trypsin EDTA for 4 minutes. The tissue was pipetted several times to make a single cell suspension and the cells were allowed to settle for 5 minutes to remove tissue clumps. For micromass culture, LBC (10 to 50 percent) were mixed with embryonic stem cells in a total of 50,000 or 100,000 cells. The mixed cells were pelleted by brief centrifugation followed by resuspension in 20 ul DMEM medium and plating on 4 well or 24 well plates. After 2 hours the cells were covered with medium. The medium was changed every other day.

### EXAMPLE 2

### Reverse Transcriptase PCR Analysis

The cells were harvested by trypsinization and total RNA was isolated. RT-PCR analysis was carried out using specific primers; for example, collagen type II, 5′-GTGAGCCATGATCCGC-3′ (SEQ ID NO: 1) and 5′-GACCAGGATTTCCAGG-3′ (SEQ ID NO: 2; Carlberg et al., 2001); oct-4, 5′-GCTTCTCTTGGAAAGGTGTTC-3′ (SEQ ID NO: 3) and 5′-sox 9, 5′-TCTTTCTTGTGCTGCACGCGC-3′ (SEQ ID NO: 4) and 5′-TGGCAGACCAGTTACCCGCATCT-3′ (SEQ ID NO: 5; Lefebvre et al., 1998); HPRT, 5′-GTAATGATCAGTCAACGGGGGAC-3′ (SEQ ID NO: 6) and 5′-CCAGCAAGCTTGCAACCTTAACCA-3′ (SEQ ID NO: 7); neomycin gene, 5′-AGGATCTCCTGTCATCTCACCTTGCTCCTG-3′ (SEQ ID NO: 8) and 5′-AAGAACTCGTCAAGAAGGCGATAGAAGGCG-3′ (SEQ ID NO: 9), at 60° C. 30 s, 72° C. 90 s, and 94° C. for 35-40 cycles. The amplified fragments were separated on 2% agarose gels.

### EXAMPLE 3

### Injection of Cells into Animals

About 1×10⁶ neo/GFP positive FVB/N embryonic stem cells exposed to precursor cells were injected into the peritoneal cavity of FVB/N mice in 100 ul of DMEM medium. The control animal was injected with 100 ul of the medium. The animals were sacrificed after about 12 weeks and tissues were collected for PCR.

### EXAMPLE 4

### Preparation and Culture of ES Cells

ES cells were cultured over monolayers of irradiated primary fibroblasts in a medium supplemented with LIF in a humidified CO₂ incubator at 37° C. following the methods described previously(Robertson, 1997). Two independent ES cell lines, R1 ES cells from the 129Sv strain of mice and FVB/N ES cells were electroporated with pEGFPN1 plasmid (Clontech, Palo Alto, Calif.), which contains the genes for neomycinphosphoribosyl transferase (neo) and enhanced green fluorescenceprotein (EGFP). The positive cells were selected with 150 mg/mlG418 and the colonies with high fluorescence activity were expanded individually. The confluent cultures of ES cells were treated with 0.25% trypsin-EDTA for 5 min and the cells were pipetted several times to obtain a single-cell suspension. The cells were allowed to stand for 10 min for feeder cells to settle, after which the top suspension of cells was collected. This was followed by another round of 10 min sedimentation to remove the feeder cells completely. Because fibroblast feeder cells are heavier than ES cells, they settle more quickly. Pure ES cells were washed two times with (Dulbecco's modified Eagle medium) DMEM containing 10% fetal bovine serum (FBS) before using them for the co-culture.

### EXAMPLE 5

### Co-Culture of ES Cells with Limb Bud

progenitor cells To obtain progenitor cells, 10.5-11.5 day pregnant FVB/N female mice were sacrificed and the limb buds from the embryos were isolated. The pooled limb buds were trypsinized in 0.25% trypsin-EDTA for 5-6 min followed by trituration to generate a single-cell suspension. The cells were washed and resuspended in DMEM with 10% FBS. The pure ES cells were co-cultured with 10%, 20%, 30%, and 40% of limb bud progenitor cells (LBPC) in high-density (about 10×10⁶ cells/ml) micro-mass cultures (Ahrens et al., 1977). The cells were mixed for 30 min on a rotator followed by centrifugation at 2000 rpm in a microfuge centrifuge. The cells were resuspended at about 10×10⁶ cells/ml, and 20-25 ml of cell suspension was transferred to 4-well Nunc plates. After 2 hr, the cells were covered with medium, which was changed every other day. After 4 days, the cells were treated with 50-100 mg/ml G418 to kill LBPC. Differentiation of cells was monitored by the appearance of condensed aggregates of cells. The differentiated cells were scraped from the plates and counted after trypsinization. Parallel cultures of pure ES cells and LBPC were used as negative and positive controls, respectively.

### EXAMPLE 6

### Alcian Blue Staining of Chondrocyte Nodules

Terminally differentiated chondrocytes express cartilage-specific sulfated proteoglycans that stain positive with alcian blue dye. After 15-17 days of culture, the cells were washed with phosphate buffered saline and fixed in 100% ethanol. The cells were then stained for 2-4 hours with alcian blue followed by washing in 100% ethanol and clarification with 80% glycerol solution. Reverse transcriptase polymerase chain reaction (RT-PCR) analysis.

### EXAMPLE 7

### Separation of Cells by Fluorescence-Activatied Cell Sorter

GFP-positive ES cells (1.5×10⁶ cells) were mixed with 500,000 LBPC (25%) isolated from a normal embryo. After thorough mixing, the cells were plated as micro-mass culture for 4 days with a change of medium every other day. The cells were harvested by trypsinization as described above and separated in fluorescent activated cell sorters.

### EXAMPLE 8

### Micro-Mass Cultures and ES Cell Differentioation

Pluripotent ES cells were co-cultured with LBPC in high-density micro-mass cultures. After 4 days, the cells were treated with G418 for up to 7 days to eliminate limb bud cells.

To induce differentiation, ES cells were co-cultured with 10%, 20%, 30%, and 40% LBPC. After about 48 hr, the co-cultured cells formed a uniform flat layer as compared to pure ES cells, which formed typical colonies of multiple cells. Within 6-7 days, or about 2-3 days after G418 treatment, aggregates of swirling cells were found dispersed throughout the culture that developed into prominent nodules after 14-15 days from the start of culture.

The morphology of the nodules was very similar to that observed in pure mesenchymal cultures. A small number of morphologically indistinct cells were also seen in the culture that might be cells that did not differentiate into chondrocytes. The nodules formed by ES cells exhibited morphology with diffused boundaries compared to that in normal mesenchymal cells. In some cultures, the nodules were densely packed with overlapping boundaries. No such nodules were observed in 14 day cultures of pure ES cells.

Chondrogenic differentiation of ES cells was observed at all concentrations (10%, 20%, 30%, and 40%) of LBPC; however, the number of nodules increased with the increase in the proportion of LBPC. For example, only 3-4 nodules were formed at 10% concentration, whereas a 5-fold and 23-fold increase was observed at 20% and 30% LBPC, respectively. A dramatic increase in nodule formation, over 250 nodules, was observed when the concentration of LBPC was raised to 40%. None of the above characteristics were observed in the control cultures of pure ES cells. Based on the above observations, 25% LBPC was used for all subsequent experiments.

As compared to 3-4 days for pure mesenchymal cells, the aggregates of cells in mixed cultures appeared after 6-8 days, or 3-4 days after G418 treatment, suggesting a lag period of about 4 days, most probably the time required for programming of the stem cells. On average, about 60%-80% of cells exhibited differentiation characteristics.

### EXAMPLE 9

### Elimination of LBPC by G418 Treatment

To investigate the effectiveness of G418 in removing the progenitor cells, cells were treated at two different stages, such as before and after the nodules were formed.

LBPC were co-cultured with ES cells for 4 days and the cells were separated in a fluorescence-activated cell sorter. The separated progenitor cells were then cultured in micro-mass culture followed by treatment with 100 mg/ml G418. Over 95% of the cells died after 7 days of G418 treatment, although a loss of about 50% of cells was observed after 5 days. The surviving cells after 7 days of treatment may be the remnants of ES cells that failed to separate by cell sorter or possibly a small percentage of cells that developed resistance to the drug. The untreated cells, on the other hand, formed only a uniform layer of cells.

In a separate experiment, micro-mass culture of freshly isolated LBPC was established. After the appearance of nodules, the cells were treated with 100 mg/ml G418. The G418-treated nodules exhibited a degenerating appearance as compared to untreated cells. These data clearly demonstrate that G418 is equally effective in killing the limb bud cells before and after they have formed nodules, supporting the formation of nodules by the differentiated ES cells in co-cultures.

### EXAMPLE 10

### Differentiation of ES Cells by LBPC

To investigate whether the induction of cell differentiation is caused by soluble factors secreted by the progenitor cells, pure ES cell cultures were treated with medium obtained from the micro-mass cultures of pure limb bud cells; the medium was changed every other day. There was no nodule formation after 14 days of culture, indicating that cell-cell interactions either alone or in combination with the soluble factors are responsible for the differentiation.

### EXAMPLE 11

### Expression of Cartilage-Specific Genes by Differentiated ES Cells

Terminally differentiated chondrocytes express genes such as collagen type II and sulfated proteoglycans specific for the extracellular matrix of cartilage. The sulfated proteoglycans stain positive with alcian blue. Nodules from co-cultured cells stained positive with the dye, similar to that observed in pure LBPC cultures. Alcian blue staining was specific to the nodules; no staining was observed in the surrounding cells. The nodules formed by the differentiated cells were densely packed with overlapping boundaries, demonstrating that almost all the nodules were formed simultaneously, indicating a programmed differentiation of ES cells.

Collagen type II is the most abundant protein in the extracellular matrix of cartilage and is expressed in two different forms due to alternate splicing of exon 2 of the MRNA. Co-cultured ES cells and LBPC were treated with 100 μg/ml G418 at day 4 to kill the limb bud-derived cells. After 3 days, or after 7 days of culture, the total RNA was isolated for RT-PCR analysis using primers that amplify DNA fragments specific for type IIA (489 bp) and type IIB (285 bp) transcripts.

Pure LBPC on the day of isolation showed exclusively amplification of a type IIA-specific 489 bp fragment, whereas the same cells at day 7 amplified DNA fragments for type IIA (489 bp) and type IIB (285 bp) transcripts, indicating differentiation into chondrocytes. On the other hand, the G418-treated co-cultures at day 7, or 3 days after G418 treatment, amplified only the type IIA-specific 489 bp fragment, indicating the pre-chondrogenic stage of the cells. No amplification of collagen type II-specific amplification was observed in ES cells at day 0 of culture. The absence of a type IIB-specific 285 bp fragment further indicates the complete elimination of LBPC by G418. However, the possibility of delayed differentiation of LBPC in the presence of ES cells cannot be ruled out.

### EXAMPLE 12

### Expression of Transcription Factor Sox9

Pre-chondrogenic cells express sry-related high-mobility group (HMG) transcription factor Sox9, which parallels the expression of collagen type II during chondrocyte differentiation. RT-PCR analysis revealed that normal LBPC at day 0 and day 7 of culture express sox9 as observed by amplification of the 135 bp fragment. The co-cultured cells at day 7, or 3 days after G418 treatment, also exhibited the expression of sox9 confirming the chondrogenic nature of the cells. The amplification of hypoxanthine phosphoribosyl transferase (HPRT) gene was used as a control for the quantity of mRNA.

### EXAMPLE 13

### Expression of ES Cell-Specific Transcription Factor Oct-4

Pluripotent ES cells express Oct-4, a POU domain specific transcription factor, whereas the expression of Oct-4 disappears rapidly as the cells differentiate into somatic cells. To observe the state of differentiation, micro-mass-cultured cells were analyzed for Oct-4 expression. Oct-4 expression decreased progressively as the cells expressed the markers of chondrocyte differentiation. RT-PCR analysis revealed that undifferentiated ES cells at day 0 showed high expression of Oct-4. After co-culture with LBPC for 7 days, Oct-4 expression decreased to about less than 10%. Oct-4, however, disappeared completely after 19 days of culture when the cells were differentiated into chondrocytes. As expected, pure LBPC did not show any expression of Oct-4.

RT-PCR analysis for HPRT was used as a control for the quantity of reverse-transcribed mRNA. No expression of chondrocyte-specific markers such as collagen type II was observed in day 0 ES cells.

What is claimed is:

1. A method of programming the differentiation of stem cells comprising:
(a) contacting stem cells with precursor cells to form a mixture, whereby the stem cells become primed; and
(b) allowing the primed stem cells to differentiate.

2. The method of claim 1 wherein the primed stem cells are introduced into an animal for in vivo differentiation.

3. The method of claim 1 wherein the primed stem cells are allowed to differentiate in vitro.

4. The method of claim 1 wherein the precursor cell is a limb bud cell.

5. The method of claim 4 wherein the differentiated cell is a chondrocyte.

6. A differentiated cell produced by the method of claim 1.

7. The method of claim 1 wherein the mixture further includes a selection compound.

8. A method to identify signals responsible for cell lineage establishment comprising:
(a) defining the prerequisite conditions to the differentiation of stem cells into their preferred lineage; and
(b) determining which prerequisite conditions are signals responsible for cell lineage establishment.

9. A method for identifying effective therapeutic agents comprising:
(a) contacting a first differentiated human stem cell with a test agent;
(b) comparing the results obtained by the test agent with the results of a control agent contacted to a second differentiated human stem cell from the same cell line.
